(19) United States
(12) Patent Application Publication
(10) Pub. No.: US 2026/0241198 A1
(43) Pub. Date: Aug. 20, 2026

(54) PHOTOTHERAPY DEVICE

(71) Applicant: DONGGUAN LAIGUANG ELECTRONIC TECHNOLOGY CO., LTD., Dongguan City (CN)

(72) Inventor: JIANGXIANG WU, Dongguan City (CN)

(73) Assignee: DONGGUAN LAIGUANG ELECTRONIC TECHNOLOGY CO., LTD., Dongguan City (CN)

(21) Appl. No.: 19/642,601

(22) Filed: Apr. 8, 2026

(30) Foreign Application Priority Data
Jan. 23, 2026 (CN) 202620096777.0

(51) Int. Cl. A61N 5/06

(52) U.S. Cl. CPC A61N 5/0616; A61N 2005/0645; A61N 2005/0654; A61N 2005/0664

(57) ABSTRACT

The present application provides a phototherapy device, which includes a flexible member, a flexible circuit board and a plurality of phototherapy lamp bodies. The flexible member has a first region and a second region, the rigidity of the second region is greater than that of the first region, and the first region is capable of flexible deformation under an external force. The flexible circuit board is built in the flexible member and deforms with the deformation of the flexible member. The plurality of phototherapy lamp bodies are integrated on the flexible circuit board and electrically connected thereto, and their positions are adjusted as the flexible circuit board deforms. At least one aperture is formed in the flexible member and provided between two adjacent phototherapy lamp bodies.

### CROSS-REFERENCE TO RELATED APPLICATIONS

The present application claims priority to Chinese patent application No. 202620096777.0, filed on January 23, 2026, the entire contents of which are incorporated herein by reference.

### TECHNICAL FIELD

The present application relates to the technical field of phototherapy devices, in particular to a phototherapy device.

### BACKGROUND

With the development of science and technology, phototherapy devices have been gradually applied in people's daily lives, and emit corresponding phototherapeutic light based on phototherapy lamp bodies, and the phototherapeutic light includes red light. The phototherapeutic light emitted by the phototherapy lamp bodies irradiates human skin. In the related art, a phototherapy device includes a base, a circuit board and a plurality of phototherapy lamp bodies, where the circuit board is connected to the base and the plurality of phototherapy lamp bodies are integrated on the circuit board. However, the base is rigid and cannot be deformed, resulting in a relatively single application scenario of the existing phototherapy devices.

### SUMMARY

The purpose of the present application is to provide a phototherapy device. A flexible member is provided with a first region and a second region, and the second region is provided on one side of the first region. The rigidity of the second region is greater than that of the first region, and the first region can undergo flexible deformation under an external force. A flexible circuit board is built in the flexible member and deforms with the deformation of the flexible member. A plurality of phototherapy lamp bodies are integrated on the flexible circuit board and electrically connected thereto, and positions of the plurality of phototherapy lamp bodies are adjusted with the deformation of the flexible circuit board. At least one aperture is formed in the flexible member and provided between two adjacent phototherapy lamp bodies. The first region can undergo flexible deformation under an external force. The plurality of phototherapy lamp bodies are integrated on the flexible circuit board and electrically connected thereto, and their positions are adjusted as the flexible circuit board deforms, to achieve a deformation effect, thereby avoiding the defect of incapability of deformation due to rigidity in the related art, the phototherapy device is adapted to different light irradiation scenarios, and the universality of the phototherapy device is improved.

To achieve the above purpose, the present application adopts the following technical scheme: a phototherapy device, including:

a flexible member, having a first region and a second region, wherein the second region is provided on one side of the first region, a rigidity of the second region is greater than that of the first region, and the first region is capable of flexible deformation under an external force;

a flexible circuit board, built in the flexible member and deforming with a deformation of the flexible member; and

a plurality of phototherapy lamp bodies, integrated on the flexible circuit board and electrically connected to the flexible circuit board;

wherein positions of the plurality of phototherapy lamp bodies are adjusted with a deformation of the flexible circuit board, and the flexible member is provided with at least one aperture, and the at least one aperture is provided between two adjacent phototherapy lamp bodies.

In one embodiment, the flexible member includes a flexible main body and a first deformable member;

the first deformable member is connected to the flexible main body and built in a part of the flexible main body where the first region is formed; and

when a user applies a deformation force to the flexible main body, the first deformable member and the first region deform under the deformation force, and the first deformable member maintains a corresponding deformed shape after being stressed.

In one embodiment, when the flexible member is rectangular, the first region is located in a long-side direction of the flexible member; and

the part of the flexible main body where the first region is formed is provided with a first receiving groove and a first cover body, the first deformable member is accommodated in the first receiving groove, and the first cover body is connected to the flexible main body and covers the first receiving groove, so that the first deformable member is covered by the first cover body.

In one embodiment, the part of the flexible main body where the first region is formed is provided with a first snap-fit groove, the first snap-fit groove is provided on one side of the first receiving groove, the first cover body is provided with a first snap-fit portion, and the first snap-fit portion is snapped into the first snap-fit groove when the first cover body is inserted into the flexible main body;

the first cover body is a deformable silicone or a deformable metal part; and

a side wall of the first cover body and an inner side wall of the first receiving groove position the first deformable member along different directions.

In one embodiment, the first deformable member is a deformable silicone or a deformable metal part.

In one embodiment, the flexible member further includes a first support member, the first support member is connected to the flexible main body and built in a part of the flexible main body where the second region is formed; and

when the user applies the deformation force to the flexible main body, original shapes of the first support member and the second region are maintained under the deformation force.

In one embodiment, the first support member is a metal plate, and a thickness of the first support member is greater than that of the first deformable member; and

under a same force condition, a forced deformation of the first support member is smaller than that of the first deformable member.

In one embodiment, when the flexible member is rectangular, the second region is located in a short-side direction of the flexible member;

the part of the flexible main body wherein the second region is formed is provided with a second receiving groove and a second cover body;

the first support member is accommodated in the second receiving groove, and the second cover body is connected to the flexible main body and covers the second receiving groove, so that the first support member is covered by the second cover body;

the part of the flexible main body wherein the second region is formed is provided with a second snap-fit groove, the second snap-fit groove is provided on one side of the second receiving groove, the second cover body is provided with a second snap-fit portion, and the second snap-fit portion is snapped into the second snap-fit groove when the second cover body is inserted into the flexible main body;

the second cover body is a deformable silicone or a deformable metal part; and

a side wall of the second cover body and an inner side wall of the first receiving groove position the first support member along different directions.

In one embodiment, the flexible member is provided with a third region, and the third region is capable of flexible deformation under an external force;

a plurality of apertures are provided along an array direction in a part of the flexible member where the third region is formed;

the part of the flexible member wherein the third region is formed is of a polymeric matrix and accommodates the flexible circuit board; and

the plurality of apertures are square holes, diamond holes, arc holes, star holes or circular holes.

In one embodiment, the flexible member is connected with a surface body, the surface body is laid on the flexible member and provided relative to the third region;

the surface body is a silicone surface body or a resin dispensing surface body;

the surface body is provided with a plurality of light-transmitting cover parts, each of the light-transmitting cover parts is provided relative to a corresponding phototherapy lamp body, such that light output by the phototherapy lamp body passes through the corresponding light-transmitting cover part; and

the phototherapy device further includes a mirror, the mirror is provided at a middle position of the third region, the mirror is mounted on the flexible member, and at least two of the light-transmitting cover parts are provided along an annular direction of the mirror.

### BRIEF DESCRIPTIONS OF THE DRAWINGS

To illustrate the technical solutions in the embodiments of the present application more clearly, the accompanying drawings required for describing the embodiments are briefly introduced below. Obviously, the accompanying drawings in the following description are only some embodiments of the present application, and those skilled in the art can obtain other accompanying drawings based on these drawings without exerting creative work.

For a more complete understanding of the present application and its beneficial effects, the following description is provided in conjunction with the accompanying drawings, in which the same reference numerals denote the same parts in the following description.

FIG. 1 is a schematic view of a phototherapy device according to an embodiment of the present application.

FIG. 2 is a first sectional view of the phototherapy device according to an embodiment of the present application.

FIG. 3 is an enlarged partial view of position A in FIG. 2.

FIG. 4 is a second sectional view of the phototherapy device according to an embodiment of the present application.

FIG. 5 is an enlarged partial view of position B in FIG. 4.

FIG. 6 is an enlarged partial view of position C in FIG. 4.

FIG. 7 is an exploded view of the phototherapy device according to an embodiment of the present application.

FIG. 8 is a schematic view of a phototherapy device according to a second embodiment of the present application.

### Reference Numerals

100: Phototherapy device;

10: Flexible member; 10a: First region; 10b: Second region; 10c: Aperture; 10d: Through hole; 10e: Third region; 11: Flexible main body; 11a: First receiving groove; 11b: First snap-fit groove; 11c: Second receiving groove; 11d: Second snap-fit groove; 12: First deformable member; 13: First cover body; 131: First snap-fit portion; 14: First support member; 15: Second cover body; 151: Second snap-fit portion; 16: Surface body; 161: Light-transmitting cover part;

20: Flexible circuit board;

30: Phototherapy lamp body;

40: Mirror

### DETAILED DESCRIPTIONS OF EMBODIMENTS

The technical solutions in the embodiments of the present application will be clearly and completely described below in conjunction with the accompanying drawings in the embodiments of the present application. Obviously, the described embodiments are only some rather than all of the embodiments of the present application. All other embodiments obtained by those skilled in the art based on the embodiments in the present application without creative work shall fall within the protection scope of the present application.

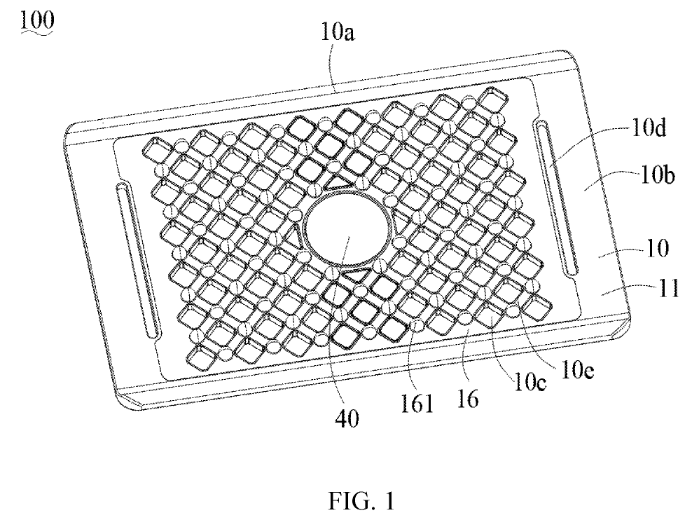

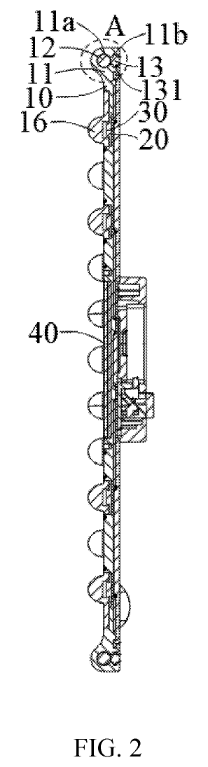

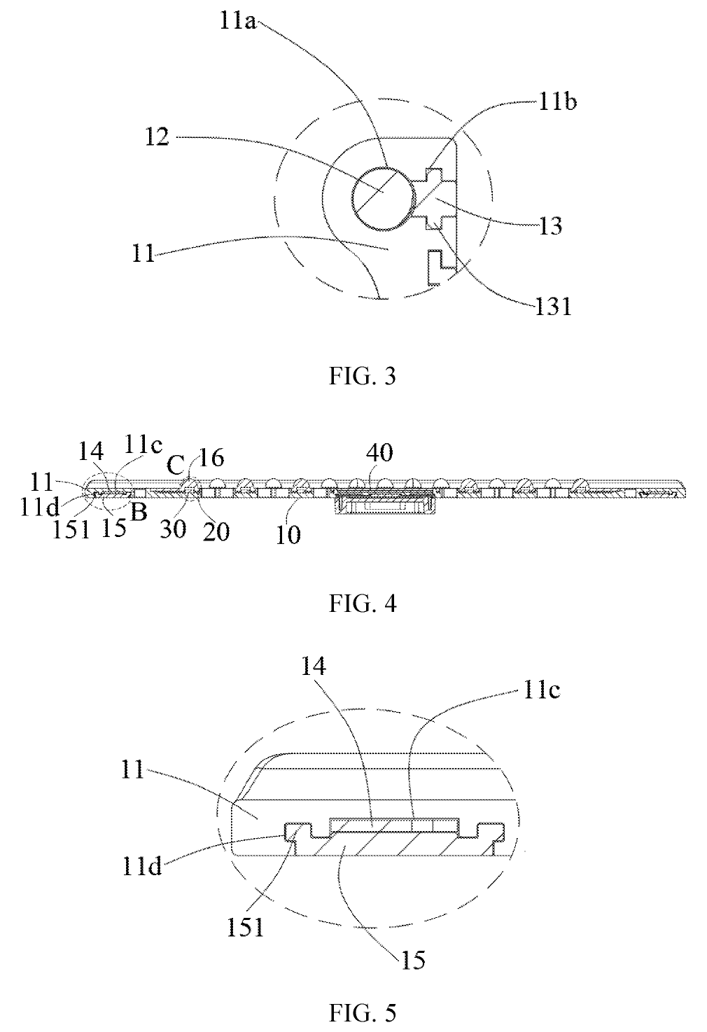

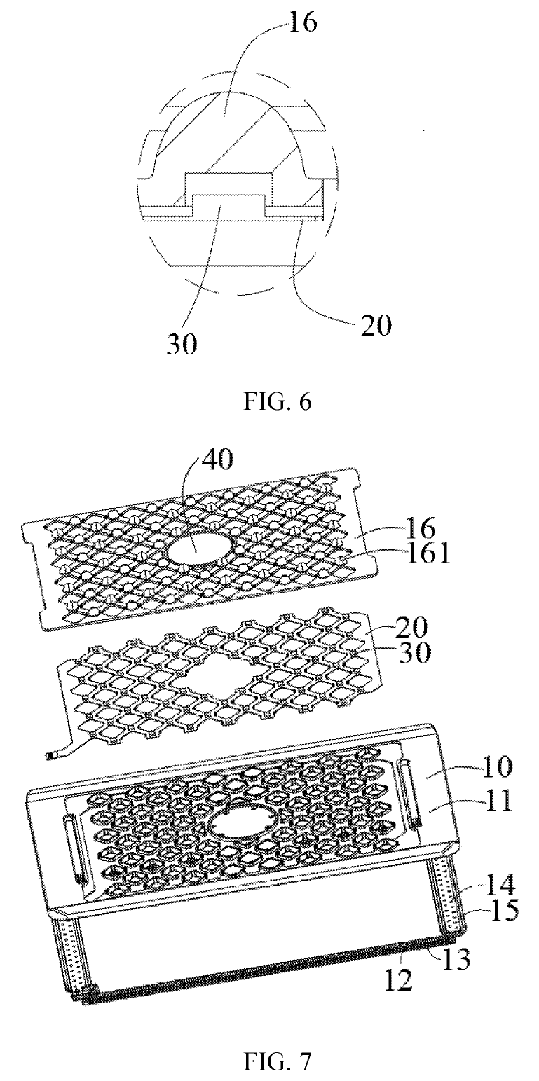

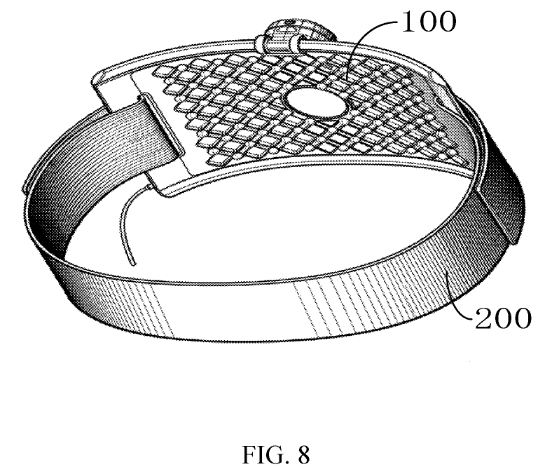

Referring to FIGS. 1 to 7, an embodiment of the present application provides a phototherapy device 100, which adopts a physical therapy method for treating diseases by light radiation.

Referring to FIGS. 1 to 7, in the embodiment of the present application, the phototherapy device 100 includes a flexible member 10, a flexible circuit board 20 and a plurality of phototherapy lamp bodies 30. The flexible member 10 has a first region 10a and a second region 10b, and the second region 10b is provided on one side of the first region 10a. The rigidity of the second region 10b is greater than that of the first region 10a, and the first region 10a can undergo flexible deformation under an external force. The flexible circuit board 20 is built in the flexible member 10 and deforms with the deformation of the flexible member 10. The plurality of phototherapy lamp bodies 30 are integrated on the flexible circuit board 20 and electrically connected thereto, and the positions of the plurality of phototherapy lamp bodies 30 are adjusted with the deformation of the flexible circuit board 20. At least one aperture 10c is formed in the flexible member 10 and provided between two adjacent phototherapy lamp bodies 30. The first region 10a can undergo flexible deformation under an external force. The plurality of phototherapy lamp bodies 30 are integrated on the flexible circuit board 20 and electrically connected thereto, and their positions adjusted as the flexible circuit board 20 deforms, to achieve a deformation effect, thereby avoiding the defects of incapability of deformation and failure to retain the deformed shape due to rigidity, the phototherapy device is adapted to different light irradiation scenarios, and the universality of the phototherapy device 100 is improved.

Referring to FIGS. 1 to 7, in the embodiment of the present application, the flexible member 10 has a first region 10a and a second region 10b, and the second region 10b is provided on one side of the first region 10a. The rigidity of the second region 10b is greater than that of the first region 10a, and the first region 10a can undergo flexible deformation under an external force, so that the flexible member 10 only deforms at the intended positions while the other regions maintain precision and strength, achieving the flexible function without sacrificing repeat accuracy and service life. The flexible circuit board 20 is built in the flexible member 10, deforms with the deformation of the flexible member 10 and retains the deformed shape, which ensures the deformation effect of the flexible circuit board 20.

The plurality of phototherapy lamp bodies 30 are provided on the side of the flexible circuit board 20 facing away from the flexible member 10 and integrated on the flexible circuit board 20, so that the plurality of phototherapy lamp bodies 30 are exposed to the external environment. The plurality of phototherapy lamp bodies 30 are electrically connected to the flexible circuit board 20, so that the flexible circuit board 20 outputs signals to the plurality of phototherapy lamp bodies 30, thereby enabling the plurality of phototherapy lamp bodies 30 to emit light. The positions of the plurality of phototherapy lamp bodies 30 are adjusted with the deformation of the flexible circuit board 20 to achieve a deformation effect, thereby avoiding the defect of incapability of deformation due to rigidity, the phototherapy device is adapted to different light irradiation scenarios, and the universality of the phototherapy device 100 is improved. Optionally, the light is red light, which is not limited herein.

At least one aperture 10c is provided in the flexible member 10 and provided between two adjacent phototherapy lamp bodies 30 to facilitate the deformation of the flexible member 10. The plurality of phototherapy lamp bodies 30 remain rigid, and the spacing and angle of the plurality of phototherapy lamp bodies 30 do not drift due to bending, ensuring the uniformity of light spots and enabling the plurality of phototherapy lamp bodies 30 to fit the curved surface of the human body smoothly.

Referring to FIGS. 1 to 7, in the embodiment of the present application, the flexible member 10 includes a flexible main body 11 and a first deformable member 12. The first deformable member 12 is connected to the flexible main body 11 and built in a part of the flexible main body 11 where the first region 10a is formed. When a user applies a deformation force to the flexible main body 11, the first deformable member 12 and the first region 10a deform under the deformation force, and the first deformable member 12 retains the corresponding deformed shape after being stressed, so that the first deformable member 12 enables the flexible main body 11 to be arbitrarily shaped like a soft strip and retain the shape without springback like a thin metal shell, achieving the effect of being fixed once bent and re-fixed upon repeated bending.

Referring to FIGS. 1 to 5, in the embodiment of the present application, when the flexible member 10 is rectangular, the first region 10a is located in a long-side direction of the flexible member 10, which ensures that the flexible member 10 can only bend along the long side without twisting or warping. No oblique torsion or wrinkles occur when the user bends the flexible member 10, and the plurality of phototherapy lamp bodies 30 remain coplanar with continuous light spots without dark areas.

The part of the flexible main body 11 where the first region 10a is formed is provided with a first receiving groove 11a and a first cover body 13, and the first deformable member 12 is accommodated in the first receiving groove 11a, so that the first deformable member 12 makes full use of the space of the first receiving groove 11a, thereby facilitating the connection of the first deformable member 12 with the flexible main body 11 through the first receiving groove 11a. The first cover body 13 is connected to the flexible main body 11 and covers the first receiving groove 11a, so that the first deformable member 12 is covered by the first cover body 13 to hide the first deformable member 12, thereby preventing the first deformable member 12 from accidental detachment, and facilitating the disassembly of the first deformable member 12.

Referring to FIGS. 1 to 3, in the embodiment of the present application, the part of the flexible main body 11 where the first region 10a is formed is provided with a first snap-fit groove 11b, the first snap-fit groove 11b is provided on one side of the first receiving groove 11a, and the first cover body 13 is provided with a first snap-fit portion 131. The first snap-fit portion 131 is snapped into in the first snap-fit groove 11b when the first cover body 13 is inserted into the flexible main body 11, so that the first cover body 13 is snapped into the flexible main body 11 through the cooperation of the first snap-fit portion 131 and the first snap-fit groove 11b, which improves the connection strength between the first cover body 13 and the flexible main body 11 and prevents the first cover body 13 from accidental detachment from the flexible main body 11.

The first cover body 13 is a deformable silicone or a deformable metal part to facilitate the deformation of the first cover body 13 with the deformation of the flexible main body 11. The side wall of the first cover body 13 and the inner side wall of the first receiving groove 11a position the first deformable member 12 along different directions, so that the first cover body 13 restricts different positions of the first deformable member 12 relative to the first receiving groove 11a, ensuring the positional accuracy of the first deformable member 12 relative to the first receiving groove 11a.

Referring to FIGS. 1 to 3, in the embodiment of the present application, the first deformable member 12 is a deformable silicone or a deformable metal part to facilitate the deformation of the first deformable member 12 with the deformation of the flexible main body 11.

Referring to FIGS. 1 to 4, in the embodiment of the present application, the flexible member 10 further includes a first support member 14 connected to the flexible main body 11 and built in a part of the flexible main body 11 where the second region 10b is formed. When the user applies a deformation force to the flexible main body 11, the first support member 14 and the second region 10b maintain their original shapes under the deformation force, so that the rigidity of the second region 10b of the flexible main body 11 is increased through the first support member 14, preventing the second region 10b from deforming with the first region 10a during the deformation of the first region 10a. The second region 10b is convenient for the user to grasp, preventing the deformation of the second region 10b when the user grasps it.

A through hole 10d is provided in the second region 10b of the flexible member 10, and the through hole 10d is used for the user's fingers to pass through, which improves the grasping convenience of the flexible member 10 for the user. In addition, the through hole 10d can also be used for a binding strap to pass through, so that the binding strap is connected to the flexible member 10, realizing the combined use of the phototherapy device and the binding strap.

Referring to FIGS. 1 to 6, in the embodiment of the present application, the first support member 14 is a metal plate, and the thickness of the first support member 14 is greater than that of the first deformable member 12, so that two elastic moduli are directly formed on the flexible member 10 through the thickness difference. Under the same force condition, the forced deformation of the first support member 14 is smaller than that of the first deformable member 12, the thick metal plate hardly moves, and the thin deformable member bends and deforms as required, so that the soft section is fixed and the hard section retains its shape instantly with a single bend by the user, without additional buckles or limiting parts.

Referring to FIGS. 1 to 7, in the embodiment of the present application, when the flexible member 10 is rectangular, the second region 10b is located in the short-side direction of the flexible member 10. A part of the flexible main body 11 where the second region 10b is formed is provided with a second receiving groove 11c and a second cover body 15, and the first support member 14 is accommodated in the second receiving groove 11c, so that the second cover body 15 makes full use of the space of the second receiving groove 11c, thereby facilitating the connection of the first support member 14 with the flexible main body 11 through the second receiving groove 11c. The second cover body 15 is connected to the flexible main body 11 and covers the second receiving groove 11c, so that the first support member 14 is covered by the second cover body 15, which hides the first support member 14, prevents the first support member 14 from accidental detachment, and facilitates the disassembly of the first support member 14.

The part of the flexible main body 11 where the second region 10b is formed is provided with a second snap-fit groove 11d, the second snap-fit groove 11d is provided on one side of the second receiving groove 11c, and the second cover body 15 is provided with a second snap-fit portion 151. The second snap-fit portion 151 is snapped into the second snap-fit groove 11d when the second cover body 15 is inserted into the flexible main body 11, so that the second cover body 15 is snapped into the flexible main body 11 through the cooperation of the second snap-fit portion 151 and the second snap-fit groove 11d, which improves the connection strength between the second cover body 15 and the flexible main body 11 and prevents the second cover body 15 from accidental detachment from the flexible main body 11.

The second cover body 15 is a deformable silicone or a deformable metal part, facilitating the installation of the second cover body 15 on the flexible main body 11. A side wall of the second cover body 15 and the inner side wall of the first receiving groove 11a position the first support member 14 along different directions, so that the first support member 14 is restricted in multiple directions, ensuring the positional accuracy of the first support member 14 relative to the flexible main body 11.

Referring to FIGS. 1 to 7, in the embodiment of the present application, the flexible member 10 is provided with a third region 10e, and the third region 10e can undergo flexible deformation under an external force, so that the third region 10e deforms with the deformation of the first region 10a, to achieve the deformation effect of the third region 10e. A plurality of apertures 10c are provided along an array direction in the part of the flexible member 10 where the third region 10e is formed, and the rigidity of the part of the flexible member 10 where the third region 10e is formed is reduced by arranging the plurality of apertures 10c. The part of the flexible member 10 where the third region 10e is formed is of a polymeric matrix and accommodates the flexible circuit board 20, so that the flexible circuit board 20 obtains the most stable mechanical environment in the softest part of the flexible member 10, which can be bent without cracking or position drift, and the service life of the flexible circuit board 20 is prolonged. The apertures 10c may be square holes, diamond holes, arc holes, star holes or circular holes, which are not limited herein.

Referring to FIGS. 1 to 7, in the embodiment of the present application, the flexible member 10 is connected with a surface body 16, which is laid on the flexible member 10 and provided relative to the third region 10e. The surface body 16 is a silicone surface body or a resin dispensing surface body, so that the surface body 16 has a deformation effect, and the surface body 16 protects the plurality of phototherapy lamp bodies 30. The surface body 16 is provided with a plurality of light-transmitting cover parts 161, each of which is provided relative to the corresponding phototherapy lamp body 30, such that the light output by the phototherapy lamp body 30 passes through the corresponding light-transmitting cover part 161, so that the plurality of light-transmitting cover parts 161 allow the light to pass through while protecting the phototherapy lamp bodies 30, thereby facilitating the conduction of the light output by the phototherapy lamp bodies 30 to the user's skin.

The phototherapy device 100 further includes a mirror 40, which is provided at the middle position of the third region 10e and mounted on the flexible member 10, and at least two light-transmitting cover parts 161 are provided along the annular direction of the mirror 40, with the mirror 40 for the user to view.

In the second embodiment of the present application, please refer to FIG. 8, a phototherapy device 100 further includes a binding strap 200, which passes through the through hole 10d and is sleeved on the user's body together with the flexible member 10, so that the phototherapy device 100 is fixed on the user's body, avoiding the user always grasping the flexible member 10 to align with the user's body during the use of the phototherapy device 100.

Compared with the related art, the beneficial effects of the present application are as follows:

The present application provides a phototherapy device 100. The flexible member 10 has a first region 10a and a second region 10b, and the second region 10b is provided on one side of the first region 10a. The rigidity of the second region 10b is greater than that of the first region 10a, and the first region 10a can undergo flexible deformation under an external force. The flexible circuit board 20 is built in the flexible member 10 and deforms with the deformation of the flexible member 10. The plurality of phototherapy lamp bodies 30 are integrated on the flexible circuit board 20 and electrically connected thereto, and the positions of the plurality of phototherapy lamp bodies 30 are adjusted with the deformation of the flexible circuit board 20. At least one aperture 10c is formed in the flexible member 10 and provided between two adjacent phototherapy lamp bodies 30. The first region 10a can undergo flexible deformation under an external force. The plurality of phototherapy lamp bodies 30 are integrated on the flexible circuit board 20 and electrically connected thereto, and their positions are adjusted as the flexible circuit board 20 deforms, to achieve a deformation effect, avoiding the defect of incapability of deformation due to rigidity, the phototherapy device is adapted to different light irradiation scenarios, and the universality of the phototherapy device 100 is improved.

It should be noted that all directional indications (such as up, down, left, right, front, back, ...) in the embodiments of the present application are only used to explain the relative positional relationship, movement state, etc. between the various components in a specific posture, and if the specific posture changes, the directional indications will change accordingly.

It should also be noted that when an element is referred to as being "fixed on" or "provided on" another element, it may be directly on the other element or an intermediate element may exist at the same time. When an element is referred to as being "connected to" another element, it may be directly connected to the other element or an intermediate element may be used for indirect connection.

In addition, the descriptions involving "first", "second", etc. in the present application are only for descriptive purposes, and cannot be understood as indicating or implying their relative importance or implicitly specifying the number of the indicated technical features. Thus, the features defined with "first" and "second" may explicitly or implicitly include at least one such feature. In addition, the technical solutions between the various embodiments can be combined with each other, but must be based on the realizability by those skilled in the art. If the combination of the technical solutions is contradictory or cannot be realized, it shall be deemed that such a combination of technical solutions does not exist and is not within the protection scope required by the present application.

The above descriptions are only the preferred embodiments of the present application, and are not intended to limit the patent scope of the present application. Any equivalent structural transformation made by using the contents of the specification and accompanying drawings of the present application under the conception of the present application, or direct/indirect application in other related technical fields shall be included in the patent protection scope of the present application.

What is claimed is:

1. A phototherapy device, comprising:
a flexible member, having a first region and a second region, wherein the second region is provided on one side of the first region, a rigidity of the second region is greater than that of the first region, and the first region is capable of flexible deformation under an external force;
a flexible circuit board, built in the flexible member and deforming with a deformation of the flexible member; and
a plurality of phototherapy lamp bodies, integrated on the flexible circuit board and electrically connected to the flexible circuit board;
wherein positions of the plurality of phototherapy lamp bodies are adjusted with a deformation of the flexible circuit board, and the flexible member is provided with at least one aperture, and the at least one aperture is provided between two adjacent phototherapy lamp bodies.

2. The phototherapy device according to claim 1, wherein the flexible member comprises a flexible main body and a first deformable member;
the first deformable member is connected to the flexible main body and built in a part of the flexible main body where the first region is formed; and
when a user applies a deformation force to the flexible main body, the first deformable member and the first region deform under the deformation force, and the first deformable member maintains a corresponding deformed shape after being stressed.

3. The phototherapy device according to claim 2, wherein when the flexible member is rectangular, the first region is located in a long-side direction of the flexible member; and
the part of the flexible main body where the first region is formed is provided with a first receiving groove and a first cover body, the first deformable member is accommodated in the first receiving groove, and the first cover body is connected to the flexible main body and covers the first receiving groove, so that the first deformable member is covered by the first cover body.

4. The phototherapy device according to claim 3, wherein the part of the flexible main body where the first region is formed is provided with a first snap-fit groove, the first snap-fit groove is provided on one side of the first receiving groove, the first cover body is provided with a first snap-fit portion, and the first snap-fit portion is snapped into the first snap-fit groove when the first cover body is inserted into the flexible main body;
the first cover body is a deformable silicone or a deformable metal part; and
a side wall of the first cover body and an inner side wall of the first receiving groove position the first deformable member along different directions.

5. The phototherapy device according to claim 2,wherein the first deformable member is a deformable silicone or a deformable metal part.

6. The phototherapy device according to claim 2, wherein the flexible member further comprises a first support member, the first support member is connected to the flexible main body and built in a part of the flexible main body where the second region is formed; and
when the user applies the deformation force to the flexible main body, original shapes of the first support member and the second region are maintained under the deformation force.

7. The phototherapy device according to claim 6, wherein the first support member is a metal plate, and a thickness of the first support member is greater than that of the first deformable member; and
under a same force condition, a forced deformation of the first support member is smaller than that of the first deformable member.

8. The phototherapy device according to claim 6 wherein when the flexible member is rectangular, the second region is located in a short-side direction of the flexible member;
the part of the flexible main body wherein the second region is formed is provided with a second receiving groove and a second cover body;
the first support member is accommodated in the second receiving groove, and the second cover body is connected to the flexible main body and covers the second receiving groove, so that the first support member is covered by the second cover body;
the part of the flexible main body wherein the second region is formed is provided with a second snap-fit groove, the second snap-fit groove is provided on one side of the second receiving groove, the second cover body is provided with a second snap-fit portion, and the second snap-fit portion is snapped into the second snap-fit groove when the second cover body is inserted into the flexible main body;
the second cover body is a deformable silicone or a deformable metal part; and
a side wall of the second cover body and an inner side wall of the first receiving groove position the first support member along different directions.

9. The phototherapy device according to claim 1, wherein the flexible member is provided with a third region, and the third region is capable of flexible deformation under an external force;
a plurality of apertures are provided along an array direction in a part of the flexible member where the third region is formed;
the part of the flexible member wherein the third region is formed is of a polymeric matrix and accommodates the flexible circuit board; and
the plurality of apertures are square holes, diamond holes, arc holes, star holes or circular holes.

10. The phototherapy device according to claim 9, wherein the flexible member is connected with a surface body, the surface body is laid on the flexible member and provided relative to the third region;
the surface body is a silicone surface body or a resin dispensing surface body;
the surface body is provided with a plurality of light-transmitting cover parts, each of the light-transmitting cover parts is provided relative to a corresponding phototherapy lamp body, such that light output by the phototherapy lamp body passes through the corresponding light-transmitting cover part; and
the phototherapy device further comprises a mirror, the mirror is provided at a middle position of the third region, the mirror is mounted on the flexible member, and at least two of the light-transmitting cover parts are provided along an annular direction of the mirror.

11. A phototherapy device, comprising:
a flexible member, having a first region and a second region, wherein the second region is provided on one side of the first region, a rigidity of the second region is greater than that of the first region, and the first region is capable of flexible deformation under an external force;
a flexible circuit board, built in the flexible member and deforming with a deformation of the flexible member; and
a plurality of phototherapy lamp bodies, integrated on the flexible circuit board and electrically connected to the flexible circuit board;
wherein positions of the plurality of phototherapy lamp bodies are adjusted with a deformation of the flexible circuit board.

12. The phototherapy device according to claim 11, wherein the flexible member comprises a flexible main body and a first deformable member;
the first deformable member is connected to the flexible main body and built in a part of the flexible main body where the first region is formed; and
when a user applies a deformation force to the flexible main body, the first deformable member and the first region deform under the deformation force, and the first deformable member maintains a corresponding deformed shape after being stressed.

13. The phototherapy device according to claim 12, wherein when the flexible member is rectangular, the first region is located in a long-side direction of the flexible member; and
the part of the flexible main body where the first region is formed is provided with a first receiving groove and a first cover body, the first deformable member is accommodated in the first receiving groove, and the first cover body is connected to the flexible main body and covers the first receiving groove, so that the first deformable member is covered by the first cover body.

14. The phototherapy device according to claim 13, wherein the part of the flexible main body where the first region is formed is provided with a first snap-fit groove, the first snap-fit groove is provided on one side of the first receiving groove, the first cover body is provided with a first snap-fit portion, and the first snap-fit portion is snapped into the first snap-fit groove when the first cover body is inserted into the flexible main body;
the first cover body is a deformable silicone or a deformable metal part; and
a side wall of the first cover body and an inner side wall of the first receiving groove position the first deformable member along different directions.

15. The phototherapy device according to claim 12,wherein the first deformable member is a deformable silicone or a deformable metal part.

16. The phototherapy device according to claim 12, wherein the flexible member further comprises a first support member, the first support member is connected to the flexible main body and built in a part of the flexible main body where the second region is formed; and
when the user applies the deformation force to the flexible main body, original shapes of the first support member and the second region are maintained under the deformation force.

17. The phototherapy device according to claim 16, wherein when the flexible member is rectangular, the second region is located in a short-side direction of the flexible member;
the part of the flexible main body wherein the second region is formed is provided with a second receiving groove and a second cover body;
the first support member is accommodated in the second receiving groove, and the second cover body is connected to the flexible main body and covers the second receiving groove, so that the first support member is covered by the second cover body;
the part of the flexible main body wherein the second region is formed is provided with a second snap-fit groove, the second snap-fit groove is provided on one side of the second receiving groove, the second cover body is provided with a second snap-fit portion, and the second snap-fit portion is snapped into the second snap-fit groove when the second cover body is inserted into the flexible main body;
the second cover body is a deformable silicone or a deformable metal part; and
a side wall of the second cover body and an inner side wall of the first receiving groove position the first support member along different directions.

18. A phototherapy device, comprising:
a flexible member;
a flexible circuit board, built in the flexible member and deforming with a deformation of the flexible member; and
a plurality of phototherapy lamp bodies, integrated on the flexible circuit board and electrically connected to the flexible circuit board;
wherein the flexible member is provided with at least one aperture, and the at least one aperture is provided between two adjacent phototherapy lamp bodies.

19. The phototherapy device according to claim 18, wherein the flexible member is provided with a third region, and the third region is capable of flexible deformation under an external force;
a plurality of apertures are provided along an array direction in a part of the flexible member where the third region is formed;
the part of the flexible member wherein the third region is formed is of a polymeric matrix and accommodates the flexible circuit board; and
the plurality of apertures are square holes, diamond holes, arc holes, star holes or circular holes.

20. The phototherapy device according to claim 19, wherein the flexible member is connected with a surface body, the surface body is laid on the flexible member and provided relative to the third region;
the surface body is a silicone surface body or a resin dispensing surface body;
the surface body is provided with a plurality of light-transmitting cover parts, each of the light-transmitting cover parts is provided relative to a corresponding phototherapy lamp body, such that light output by the phototherapy lamp body passes through the corresponding light-transmitting cover part; and
the phototherapy device further comprises a mirror, the mirror is provided at a middle position of the third region, the mirror is mounted on the flexible member, and at least two of the light-transmitting cover parts are provided along an annular direction of the mirror.
